(19) United States
(12) Patent Application Publication
(10) Pub. No.: US 2019/0083588 A1
(43) Pub. Date: Mar. 21, 2019

(54) METHOD FOR TREATING AXONS WITH BOTULINUM TOXIN

(71) Applicant: Colin K. Franz, Chicago, IL (US)

(72) Inventor: Colin K. Franz, Chicago, IL (US)

(21) Appl. No.: 16/131,171

(22) Filed: Sep. 14, 2018

Related U.S. Application Data
(60) Provisional application No. 62/558,739, filed on Sep. 14, 2017.

(51) Int. Cl. A61K 38/48; A61P 25/00; A61K 9/00

(52) U.S. Cl. CPC A61K 38/4893; A61K 9/0085; A61K 9/0019; A61P 25/00

(57) ABSTRACT

This disclosure relates to methods and compositions for inducing growth of peripheral nervous system neurons using an effective amount of a botulinum toxin. The disclosure also relates to methods for the treatment of a patient in need thereof such as a patient undergoing nerve transfer surgery, the method comprising administering to the patient an effective amount of a botulinum toxin.

This application claims priority to U.S. Provisional Application No. 62/558,739, the disclosure of which is explicitly incorporated by reference herein.

### FIELD OF THE INVENTION

This disclosure relates to methods and compositions for inducing growth of peripheral nervous system neurons using an effective amount of a botulinum toxin. The disclosure also relates to methods for the treatment of a patient in need thereof such as a patient undergoing nerve transfer surgery, the method comprising administering to the patient an effective amount of a botulinum toxin.

### BACKGROUND

Peripheral nerve injury (PNI) is a common cause of functional impairment [1, 2]. The causes of PNI are varied, ranging from penetrating trauma in combat to chronic compressive states such as carpal tunnel syndrome. Key to achieving optimal functional outcome after PNI is to minimize the duration of target denervation [3, 4]. To this end Gordon and Chan have been working to translate a brief electrical stimulation protocol that is applied directly to cut axons [5, 6], but despite their promising initial clinical trial there remains significant barriers to broader application of this approach. For example, the lack of clinician remuneration for delivering this added service, increased time in the operating room to apply stimulation therapy, and/or coordination of the post-operative transfer of patient to a specialty neurophysiology laboratory for both stimulation treatment and subsequent removal of wires electrodes.

Even prior to Gordon's brief electrical stimulation protocol, the conditioning lesion effect (CLE) is one of the most well studied and robust experimental strategies to enhance axon regeneration [7]. Briefly, the classic CLE paradigm results in accelerated axonal regeneration following an axotomy (the testing lesion) as a result of the axon having undergone a previous injury (the conditioning lesion) 1 to 2 weeks earlier [8, 9]. It is widely believed that the CLE is largely attributable to early neuron intrinsic changes after denervation [7], but it has generally viewed as infeasible for clinical translational based on its invasiveness and the temporal requirement for its application (i.e. prior to nerve injury). To the second point, while a pre-conditioning treatment is generally not possible when a traumatic PNI is surgically managed solely with a primary repair (e.g. suturing a lacerated nerve back onto itself), many peripheral nerve surgeries now involve a nerve transfer procedure given recent reports of superior clinical outcomes [10]. Nerve transfer surgery consists of the isolation and redirection of a functionally redundant, healthy donor nerve fascicle(s) located in close proximity to the denervated muscle targets of the damaged nerve with the goal to achieve the earliest muscle reinnervation possible [11]. Since the donor nerve has intact neuromuscular connections prior to transfer, it is amendable to a pre-conditioning treatment.

### SUMMARY OF THE INVENTION

The disclosure provides a method of inducing growth of a peripheral nervous system neuron, the method comprising administering to the neuron an effective amount of a botulinum toxin.

In particular embodiments, the peripheral nervous system neuron is a motor neuron. In particular embodiments, the neuron has been damaged or axotomized.

In particular embodiments, the neuron growth comprises neurite outgrowth or neuronal regeneration.

In particular embodiments, the neuron is in a mammal such as a human.

In particular embodiments, the effective amount of the botulinum toxin is 0.1 units (4.4-5.1 units/kg), 0.25 units (11.0-12.75 units/kg) or 0.5 units (22.0-25.5 units/kg). In particular embodiments, the effective amount of the botulinum toxin is 0.25 units (11.0-12.75 units/kg).

In particular embodiments, the botulinum toxin is administered to the neuron by injection or administered topically to the neuron. In particular embodiments, the botulinum toxin is administered to the cell body of the neuron. In particular embodiments, the botulinum toxin is administered the axon of the neuron.

In particular embodiments, the botulinum toxin is type A neurotoxin.

The disclosure also provides a method of inducing neuronal growth in a patient in need thereof, the method comprising administering to the patient an effective amount of botulinum toxin.

In particular embodiments, the patient is undergoing a nerve transfer surgery. In particular embodiments, the botulinum toxin is administered to the patient one week prior to surgery.

### DESCRIPTION OF THE FIGURES

FIG. 1 displays a visual diagram reflecting experimental design for mouse tibial nerve injury model. In FIG. 1A, intramuscular BoTX-A injection (1) was performed into triceps surae. One week later the tibial nerve was crushed (2). In FIG. 1B, one week after nerve crush surgery a nerve biopsy was taken distal to the injury site (3) and fluorescent tracer was applied (4).

FIG. 2 displays study results relating to dosing of BoTX-A injection. In FIG. 2A, the SHAM condition received a saline vehicle only injection and the murine demonstrates a normal toe spreading response. In FIG. 2B, the 0.25 U of BoTX-A condition impaired murine to spreading response 3 days after injection (black arrow). In FIG. 2C, the Digit Abduction Score showed at the 0.25 and 0.50 U doses there was the most severe paresis that recovered to baseline over 3-4 weeks.

FIG. 3 displays study results indicating that BoTX-A injection induces neuromuscular sprouting. In FIG. 3A, two weeks following 0.25 U BoTX-A injection, terminal axon sprouting was induced. In FIG. 3B, controls neuromuscular junctions rarely had terminal sprouts (white arrows). In FIG. 3C, significantly more terminal sprouts were identified after BoTX-A injection. Scale bar represents 10 μm. *, represents P<0.01.

FIG. 4 indicates that BoTX-A conditioning increases axonal reinnervation. One week following BoTX-A injection more myelinated axons were observed in the distal nerve stump then after control tibial nerve injury (FIG. 4A and FIG. 4B). This represents a significantly increased number of myelinated axon profiles in BoTX as compared to SHAM control (FIG. 4C). Scale bar represents 5 μM.*, represents P=0.004.

FIG. 5 indicates that BoTX-A conditioning increases motor reinnervation. FIG. 5A is an image reflecting that one week following fluorescent tracer application motor neurons had reinnervated the distal nerve at different time points were easily quantifiable. FIG. 4B is a bar chart reflecting the number of retrogradely labelled motor neurons 1 or 4 weeks after tibial nerve crush. Scale bar represents 100 μM. *, represents P=0.02.

FIG. 6 reflects that BoTX-A conditioning increases hESC-derived MN neurite growth. FIG. 6A is a visual representation of treatments and timing of human MN neurite regrowth assay. FIG. 6B is a bar chart reflecting spontaneous electrical of MN cultures at two weeks (baseline) and 2-4 days following BoTX-A or SHAM treatments. FIG. 6C is a bar chart reflecting LDH cytotoxicity assay performed 2 days after treatment and did not reveal a difference between BoTX-A vs. SHAM controls. FIGS. 6D and 6E are representative photomicrographs of SHAM and BoTX-A treated MNs. FIG. 6F is a bar chart reflecting proportion of neurons with neurites at endpoint. FIG. 6G is a bar chart reflecting mean number of neurites per neuron at endpoint. FIG. 6H is a bar chart reflecting the mean length of the longest neurite formed per neuron at endpoint. Scale bar represents 10 μM. *, represents P<0.001.

The patent or application file contains at least one drawing executed in color. Copies of this patent or patent application publication with color drawing(s) will be provided by the Office upon request and payment of the necessary fee.

### DETAILED DESCRIPTION

This disclosure relates to methods and compositions for inducing growth of peripheral nervous system neurons using an effective amount of a botulinum toxin. The disclosure also relates to methods for the treatment of a patient in need thereof such as a patient undergoing nerve transfer surgery, the method comprising administering to the patient an effective amount of a botulinum toxin.

As utilized in accordance with the present disclosure, the following terms, unless otherwise indicated, shall be understood to have the following meanings. Unless otherwise required by context, singular terms shall include pluralities and plural terms shall include the singular.

In particular embodiments provided herein are methods of inducing growth of a peripheral nervous system neuron, the methods comprising administering to the neuron a botulinum toxin.

As used herein, “inducing growth of a neuron” refers to an increase in the rate or degree of the development of an embryonic neuron, the continued development of a young neuron, the regeneration of any part of a damaged neuron, or any change in size, shape, or structure of a viable neuron. In particular embodiments, neuronal growth includes neurite outgrowth which is the development and extension or elongation of any projections from the cell body of a neuron. In other embodiments, neuronal growth includes neuronal regeneration. As used herein “neuronal regeneration” refers to renewal or physiological repair of damaged nerve tissue.

Botulinum toxin (BoTX) is a neurotoxin protein naturally produced by various bacteria, e.g., Clostridium botulinum. At least eight different serotypes of BoTX are recognized, and they are commonly designated as A, B, C1, C2, D, E, F, and G. Exemplary sources of BoTX are C. argentinense, C. baratii, C. botulinum, and C. butyricum. In one embodiment, the BoTX is a type A neurotoxin. The term BoTX as used herein includes pieces, portions and fragments of the neurotoxic protein that retain neurotoxin activity. In addition to fragments, BoTX also refers to complexes that include the neurotoxin. The BoTX may in a pure state, e.g., free from other proteins, or it may be in combination with or in complex with other proteins. The BoTX may be isolated from bacteria or it may be obtained through chemical synthesis, or it may be produced recombinantly.

In particular embodiments, the neuron is a motor neuron. In particular embodiments, the neuron has been damaged or has been axotomized.

In particular embodiments, the neuron is from a mammal such as a human.

The term “effective amount” refers to an amount or dosage sufficient to produce a desired result. The effective amount may vary depending on the botulinum toxin that is being used, and may also depend on a variety of factors and conditions related to the patient being treated and the severity of the disorder. For example, if the botulinum toxin is to be administered in vivo, factors such as the age, weight, and health of the patient as well as dose response curves and toxicity data obtained in preclinical animal work would be among those factors considered. The determination of an effective amount is within the ability of those skilled in the art. In particular embodiments the botulinum toxin is administered at a concentration of 0.1 units (4.4-5.1 units/kg), 0.25 units (11.0-12.75 units/kg) or 0.5 units (22.0-25.5 units/kg). In particular embodiments, the effective amount of the botulinum toxin is 0.25 units (11.0-12.75 units/kg).

The botulinum toxin may be adapted for direct topical application to exposed neurons or for administration to non-exposed neurons by indirect routes including intramuscular, intravenous or intraperitoneal administration. In particular embodiments, the botulinum toxin is administered to the cell body of the neuron. In other embodiments, the botulinum toxin is administered to the axon of the neuron.

In a particular embodiments, provided herein are methods of inducing neuronal growth in a patient in need thereof, the methods comprising administering to the patient an effective amount of a botulinum toxin.

As used herein the term “patient” refers to any individual who is the target of administration. The patient can be, for example, a mammal. Thus, the patient can be a human. Those patients in need of treatment using botulinum toxin include those having nerve damage for example peripheral nerve injuries, plexopathies, and spinal nerve root avulsions. In particular embodiments, botulinum toxin can be used to promote neuronal growth in patients wherein the patient is undergoing a nerve transfer surgery. Peripheral nerve transfer surgeries are also routinely being offered to patients with spinal cord injury [50] as well as recently published clinical trial suggested nerve transfer surgery may improve arm function in patients with spastic paralysis from a cortical lesion [51].

Dosing frequency of the botulinum toxin will depend upon the botulinum toxin in the formulation being used. Typically, a clinician will administer the botulinum toxin until a dosage is reached that achieves the desired effect. The composition can therefore be administered as a single dose, as two or more doses (which may or may not contain the same amount of the desired molecule) over time, or as a dose. In particular embodiments, the botulinum toxin is administered to the patient prior to the surgery. In particular embodiments, the botulinum toxin is administered to the patient 1, 2, 3, 4, 5, 6, or 7 days prior to surgery. In particular embodiments, the botulinum toxin is administered to the patient 1, 2, 3, 4, 5 6, or 7 weeks prior to surgery.

### EXAMPLES

The Examples that follow are illustrative of specific embodiments disclosed herein and various uses thereof. They are set forth for explanatory purposes only and are not to be taken as limiting.

### Example 1: Materials and Method

### Mice

Young adult (8-12 week old) male C57Bl/6 mice were obtained from Jackson Labs (Bar Harbor, Me.). Mice were maintained in a 12/12 light/dark cycle with ad libitum access to food and water.

### BoTX-A Injections

Mice were injected with OnabotulinumtoxinA (BoTX-A; Allergan, Irvine, Calif.) into the triceps surae muscle group on the right side. For dose response experiments, mice were injected in the triceps surae muscle group with 0.1 units (4.4-5.1 units/kg), 0.25 units (11.0-12.75 units/kg) or 0.5 units (22.0-25.5 units/kg) of BoTX diluted to a final volume of 50 μl. The Sham group received a volume matched (50 μl) injection of saline vehicle. All injections were performed in transcutaneous fashion with a 31 gauge needle attached to a 0.3 mL insulin syringe (BD Medical, Franklin Lakes, N.J.) targeted at a single site in the upper ⅓ of the posterior compartment of the lower leg at midline such as to block the triceps surae muscles. Based on these results, a dose of 0.25 units (11.0-12.75 units/kg) was selected as the ideal treatment dose in subsequent axon regeneration experiments.

### Surgery

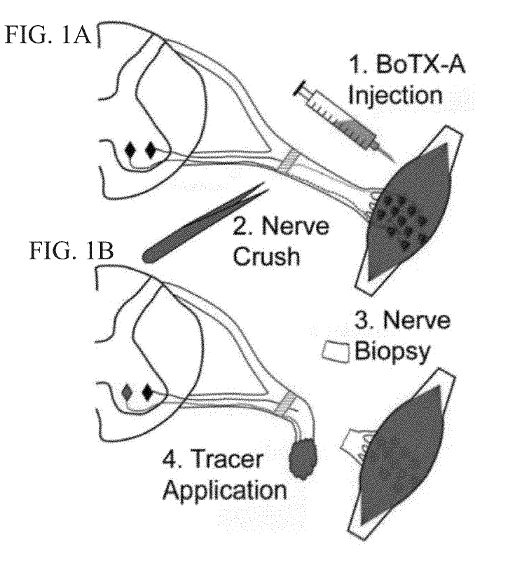

Mice were anesthetized with 1-2% isoflurane and surgery was performed under aseptic conditions. An incision was made parallel to and below the femur to expose the sciatic nerve and then the tibial nerve branch was isolated. The tibial nerve was crushed with #3 jeweler's forceps (FIG. 1 A) holding down for 10 seconds with visual confirmation that all axons were severed as indicated by a resultant translucent appearance of the nerve segment. This was repeated once more for an additional 10 seconds to ensure all axons were transected. The crush site was marked with an 11-0 suture (Fine Science Tools) carefully tied through the epineurium so the site could be readily identified.

### Retrograde Labeling of Motor Neurons

After either 1 or 4 weeks, a second operation was performed to assess the number of motor neurons that had reinnervated the distal nerve. The tibial nerve was transected 10 mm distal to the crush site (identified by 11-0 suture knot) and 10% Fluoro-ruby dye (DS-1817, Thermo-Fisher Scientific, Waltham, Mass.) prepared in sterile saline was applied to the distal nerve with Gel Foam (Pfizer, New York City, N.Y.) as previously described [18] (FIG. 1B). On day 7 after tracer application, mice were transcardially perfused with 4% paraformaldehyde (PFA) prepared in phosphate buffered 0.9% saline (PBS). The lumbosacral enlargement of the spinal cord was isolated and dissected free of the vertebral column, post-fixed in 4% PFA solution overnight, cryoprotected in 30% sucrose in PBS, flash frozen, and then sectioned on a cryostat. Spinal cords were sectioned in sagittal orientation at 60 μm thickness. Retrogradely labeled motor neurons were counted as described previously [19] and raw counts were corrected by the Abercrombie method [20]. All cell counts were performed by an observer blinded to treatment groups.

### Myelinated Axon Counts

The 2 mm segment of tibial nerve immediately distal to the site of retrograde labeling (described above) was biopsied at the time of the second operation (FIG. 1B). Biopsy samples were fixed in 0.1 M sodium cacodylate buffer pH7.3 containing 2% paraformaldehyde and 2.5% glutaraldehyde and post-fixed with 2% osmium tetroxide in 0.1M sodium cacodylate buffer, rinsed with distilled water, en bloc stained with 3% uranyl acetate, rinsed with distilled water, dehydrated in ascending grades of ethanol, transitioned with propylene oxide and embedded in resin mixture of Embed 812 kit, cured in a 60° C. oven. Samples were sectioned on a Leica Ultracut UC6 ultramicrotome (Buffalo Grove, Ill.) at 1 um thickness and were collected and stained with Toluidine Blue-O. Samples were processed by the Cell Imaging Facility (core) at Northwestern University Feinberg School of Medicine. Images were captured on a Leica DM2500 LED upright microscope. Myelinated axons counts were made by a blinded observer using Image J (National Institute of Mental Health, Bethesda, Md.).

### Behavioral Analysis

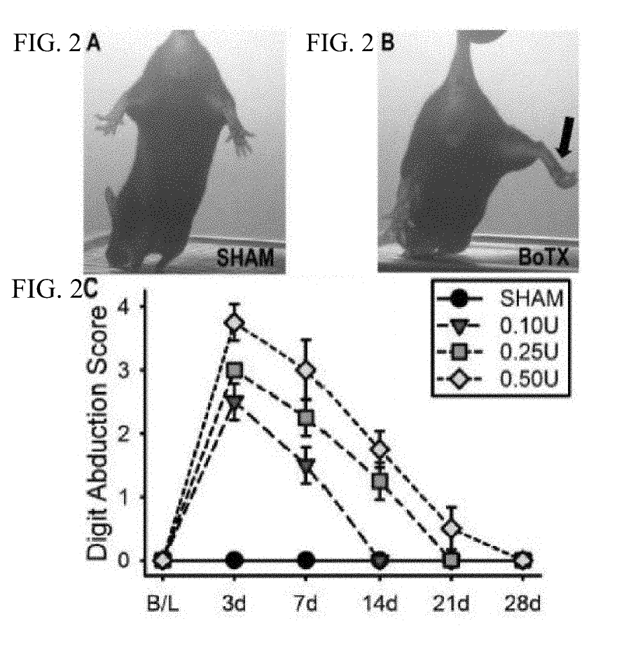

For the BoTX-A dosing experiments the Digit Abduction Score (DAS) assay was performed, which is an observational evaluation of the BoTX-A-induced paresis as described by Aoki [21]. Briefly, a startle response was elicited by suspending mice hind limbs by grasping the tail, which produces a stereotypical reaction in which the mouse abducts its hind digits (FIG. 2A). Varying degrees of digit abduction were scored on a five-point scale (0=normal to 4=maximal reduction in digit abduction and leg extension) by an observer who was masked to treatment immediately before injection, then 3, 7, 14 and 21 days post-injection. Mice were injected in the triceps surae muscle group at different doses as outlined above.

### Neuromuscular Junction Staining

The triceps surae muscle group was isolated and dissected from the right leg of a PFA-perfused mouse, immersed in 20% sucrose mixed with OCT (1:2), pinned at its physiological resting length, flash frozen in dry-ice-cooled isopentane, and the mid-belly of its proximal compartment was cryostat sectioned on to slides at 20 μm thickness. The tissue was air dried overnight, blocked for 1 hr at room temperature in PBS containing 0.3% triton x-100 and 2.5% bovine serum albumin (BSA), incubated overnight at 4° C. in rabbit anti-neurofilament SMI-312 (1:1000; ab24574, Abcam, San Francisco, Calif.) diluted in blocking solution, washed several times with PBS, incubated overnight at 4° C. in goat anti rabbit IgG secondary antibody conjugated to Alexa Fluoro 488 (1:500; ThermoFisher Scientific, Waltham, Mass.) and rhodamine conjugated α-bungarotoxin (1:100; ThermoFisher Scientific) diluted in blocking solution, washed several times in PBS, and then mounted in ProLong Gold mounting media (ThermoFisher Scientific). Images were acquired with Leica DM2500 LED upright microscope as described above for motor neurons. The percentage of innervated neuromuscular junctions was determined based on the co-localization of the pre-synaptic (neurofilament) and postsynaptic (α-bungarotoxin) markers.

### Culturing of Human Stem Cell Derived Motor Neurons

Motor neuron (MN) differentiation was carried out as previously described [22] on a human embryonic stem cell (hESC) line (HUES 64) that was obtained from Harvard Stem Cell Science Core facility. Briefly, hESC colonies were dissociated to single cells with Accutase (Sigma-Aldrich, St. Louis, Mo.) and plated in suspension in low-adherence flasks, at a 400K/ml density with 10 μM ROCK inhibitor (Sigma-Aldrich) in mTeSR1 media for 24 hrs. Embryoid bodies (EBs) were formed and media was gradually diluted (50% on day 3 and 100% on day 4) to KOSR (DMEM/F12, 15% KOSR) between days 1-4 and to a neural induction medium (NIM: DMEM/F12 with L-glutamine, NEAA, Heparin (2 μg/ml), N2 supplement (ThermoFisher Scientific) for days 5-24. Treatment with small molecules and recombinant proteins was as follows: on d1-d6, 10 μM SB431542 (Sigma-Aldrich)+1 μM Dorsmorphin (Stemgent, Lexington, Mass.); on d5-d24 10 ng/mL BDNF (R&D Biosystems, Minneapolis, Minn.), 0.4 μg/ml ascorbic acid (Sigma-Aldrich), 1 μM Retinoic Acid (Sigma-Aldrich) and 1 μM Smoothened Agonist 1.3 (Calbiochem, Billerica, Mass.). At day 24 EBs were dissociated to single cells with Papain/DNase (Worthington Bio, Lakewood, N.J.) and frozen for future use. MNs were thawed and plated as single cells onto surfaces coated with Poly-D-Lysine (PDL; 0.1 mg/mL; BD Biosciences, San Jose, Calif.) and Laminin (20 μg/mL; BD Biosciences) for motor axon re-growth experiments.

### Motor Neuron Neurite Assay and BoTX-A Treatment

Initially, 500K differentiated MN cultures were plated on 12-well cell culture plates (ThermoFisher Scientific) coated with PDL/Laminin [22]. MN cultures were maintained in Neurobasal media (ThermoFisher Scientific), supplemented with B27 and N2 supplement (ThermoFisher Scientific), 10 ng/mL of each of BDNF, GDNF, CNTF (R&D Biosystems) and 0.4 μg/ml ascorbic acid (AA; Sigma-Aldrich) and fed every 2-3 days. After 14 days, MN cultures were treated with 1 ml of 2 U/ml BoTX-A or a vehicle control, allowed to incubate for 24 hours before changing the media, and then fed every other day until 1 week post BoTX-A treatment. At this point, day 21, MN cultures were dissociated to single cells with Accutase and 40K were replated onto 10 mm glass coverslips in a 24-well cell culture plate and grown as above in NBM plus each of BDNF, GDNF, CNTF, AA. Of note, no ROCK inhibitor was added for this replating step. After 24 hours they were then fixed in 4% PFA for 20 minutes and then switched to PBS solution. To evaluate MN neurite morphology fixed cultures were blocked for 1 hr at room temperature in PBS containing 0.3% triton x-100 and 2.5% bovine serum albumin (BSA), incubated overnight at 4° C. in rabbit anti-neuronal class III β-tubulin (1:1000; PRB-435P, Covance, Emeryville, Calif.) and mouse anti-islet ½ (1:100; DSHB, Iowa City, Iowa) diluted in blocking solution, washed several times with PBS, incubated overnight at 4° C. in goat anti-rabbit IgG secondary antibody conjugated to Alexa Fluoro 488 (1:500; Invitrogen) and goat anti-mouse IgG secondary conjugated to Alexa Fluoro 555 (1:500; Invitrogen) diluted in blocking solution, washed several times in PBS, and then mounted in ProLong Gold mounting media with DAPI (ThermoFisher Scientific). Images were acquired with Leica DM2500 LED upright microscope. Neurite measurements were made using Image J (National Institute of Mental Health) and performed by an assessor blinded to treatment conditions.

### Multi-Electrode Array Recordings of MN Cultures

In some cases, 20K hESC-derived MNs were plated on 48-well multielectrode (MEA) plates with 16 extracellular electrodes/well for recordings of spontaneous neural activity on the Maestro (Axion BioSystems) MEA recording amplifier with a head stage that maintained a temperature of 37° C. The MEA data was sampled at 15 kHz, digitized, and analyzed using Axion Integrated Studio software (Axion BioSystems) with a 200 Hz high pass and 2500 kHz low pass filter and an adaptive spike detection threshold set at 5.5 times the standard deviation for each electrode with 1 second binning. These standard settings were maintained for all Axion MEA recording and analysis. Cell survival was measured with the CytoTox 96® Non-Radioactive Cytotoxicity Assay according to manufacturer's protocol (Promega, Madison, Wis.).

### Statistics

Mean values(±standard error of the mean; SE) are shown throughout. The Student's t-test was used to make comparisons between time-matched BoTX-A and SHAM data.

### Example 2: Induction of Transient Muscle Paresis and Neuromuscular Sprouting with BoTX

The effect of BoTX-A injection unilaterally into the triceps surae muscle group was examined over a 4 week period using the Digit Abduction Score (DAS) motor behavior assay [21] (FIG. 2A-B) that evaluates for the toe spreading reflex. A dose dependent pattern in the DAS score with increasing concentrations of toxin leading to decreased digit abduction relative to vehicle treated control was demonstrated (FIG. 2C). The peak DAS score for all BoTXA doses (0, 0.1 0, 0.25 and 0.50 U/mouse) studied was observed on day 3 post-injection and had already began to decline by day 7. By day 14, the DAS scores from the 0.10 U dose group had returned to baseline, and by day 21, the DAS scores from the 0.25 and 0.50 U dose groups returned to baseline as well. Since 0.25 and 0.50 U doses were equivalent, all subsequent in vivo experiments were based on a dose of 0.25 U/mouse.

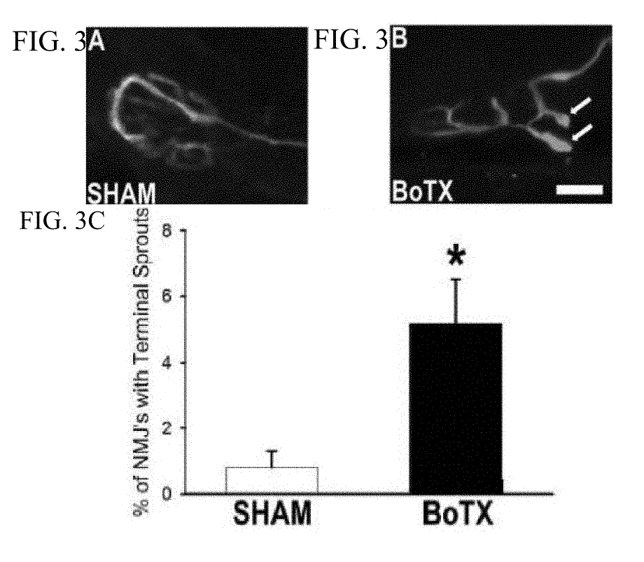

To assess for terminal sprouting from chemo-denervated neuromuscular junctions BoTX-A was injected at a dose of 0.25 U into the triceps surae muscle group (FIG. 3A-B). Two weeks following the injection the muscle was harvested and frozen sections were stained for presynaptic (neurofilament) and post-synaptic (acetylcholine receptors). Terminal sprouts were (FIG. 3B) present at 5.2±1.3% (n=5 mice, m=250 endplates) in the BoTX-A treated condition, whereas just 0.8±0.5% (n=5 mice, m=250 endplates) in the SHAM treated condition (FIG. 3C; p<0.01).

### Example 3: BoTX Pre-conditioning Enhances Motor Nerve Reinnervation

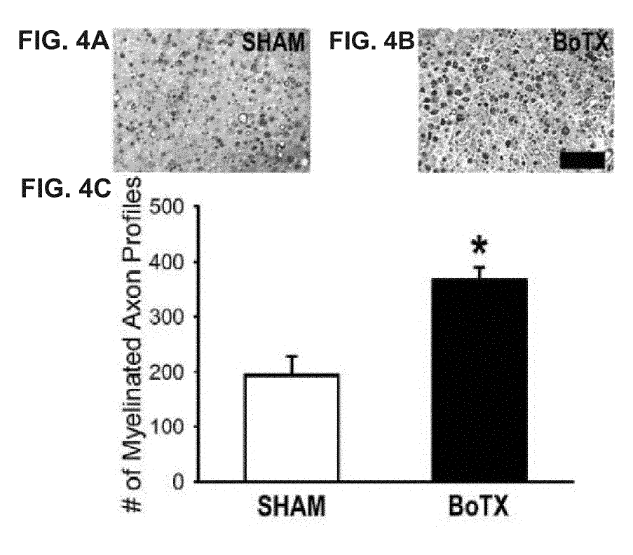

This example investigated the effect of BoTX-A pre-conditioning on motor axon regeneration. The pre-conditioning dose of 0.25 U was injected unilaterally into the triceps surae muscle group 1 week before a tibial nerve crush injury was performed (FIG. 1A). Toluidine blue staining was performed on nerve biopsies to compare the number of myelinated axons that had reinnervated the distal tibial nerve 1 week following nerve crush. Qualitative assessment of the stained nerve biopsied revealed a consistent pattern where there appeared to be a greater density of myelinated axons in the nerves that had been pre-treated with BoTX-A (FIG. 4A-B). To accurately quantitate this observation, blinded manual counts of the number of myelinated fibers that had reinnervated each nerve sample were performed. The number of myelinated axon profiles counted 10-12 mm distal to the site of nerve crush in the BoTX-A (366±24, n=5) versus SHAM (193±36, n=5) was significantly enhanced (P=0.004; FIG. 4C-D).

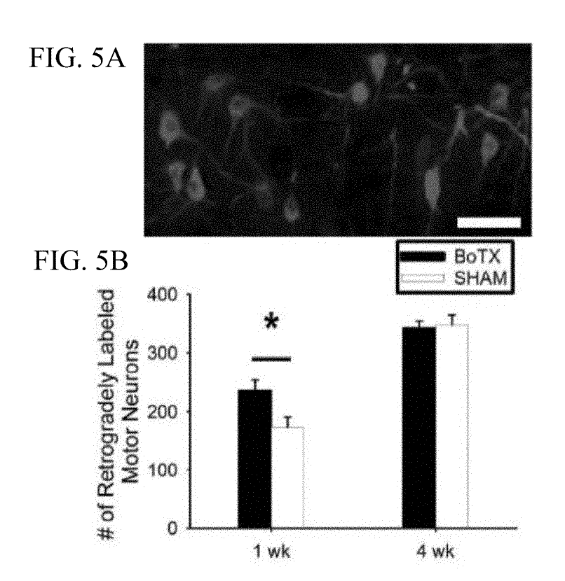

To examine the effects of BoTX-A pre-conditioning on motor reinnervation directly the number of retrogradely labeled MNs that picked up Fluoro-ruby dye applied 10 mm distal to the repair were counted (FIG. 1B). This technique was previously [18] demonstrated to effectively label MNs in the ventral spinal cord that are quantifiable (FIG. 5A). Blinded manual counts of the number of motor neurons labeled in each treatment group after 1 or 4 weeks were performed. The number of labeled MNs in the BoTX-A (236±18, n=11) versus the SHAM (172±19, n=10) group was significantly higher at 1 week (P=0.02; FIG. 5B). By 4 weeks of recovery, when the number of regenerating MNs was expected to plateau, there was no difference in the number of labeled MNs between the BoTX-A (342±12, n=3) versus the SHAM (347±18, n=3) group (p=0.83; FIG. 5B).

### Example 4: BoTX Pre-Conditioning Enhances Human Motor Neuron-Neurite Outgrowth

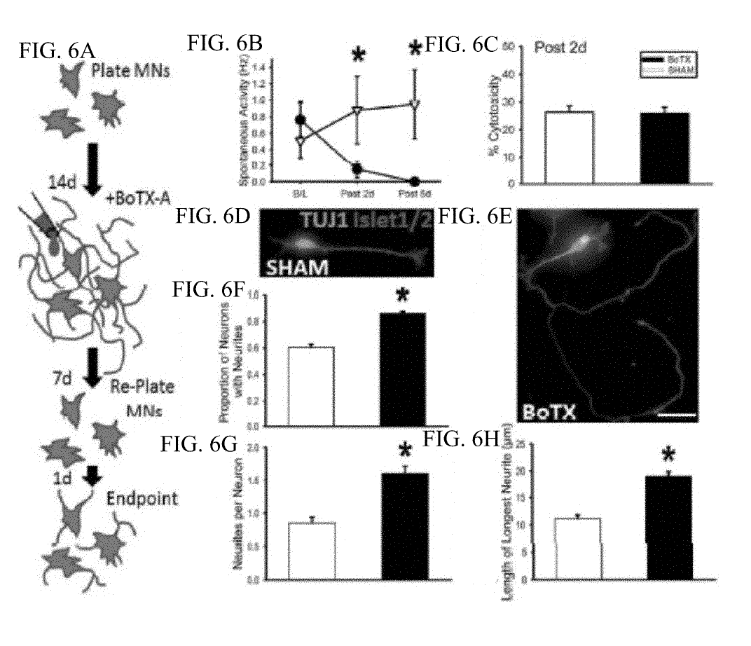

Given the substantial effects from BoTX-A pre-conditioning on motor axon regeneration in mice, this exampled investigated whether a similar effect would be seen in a human preclinical model of MN-neurite outgrowth. To block neurotransmission in this culture system BoTX-A was applied two weeks after plating MNs (FIG. 6A). Treatment with BoTX-A at 2 weeks blocked neural activity within 48 hrs (FIG. 6B). It did so without causing any detectable change in neuron viability (BoTX-A 25.9±2.3% vs. SHAM 26.3±2.4% cytotoxicity, n=9/group) as assessed by lactate dehydrogenase (LDH) levels, a stable cytosolic enzyme that is released upon cell lysis.

BoTX-A or saline-vehicle SHAM pre-conditioning was applied to human ESC derived MN cultures on Day 14. Analogous to the in vivo paradigm, following one additional week (i.e. Day 21) detachment and then re-plating the human ESC-derived MNs on a PDL-Laminin coated glass coverslip was performed. Plated human ESC-derived MNs were allowed to grow for 24 hours, since this early time point was associated with limited outgrowth for untreated ESC-derived MNs. MNs were analyzed in the BoTX-A (166 cells; 5 replicates) and SHAM (186 cells; 4 replicates) groups for their proportion with initial process formation (neurite initiation) (FIG. 6F), the number of neurites per neuron with a neurite process equal to or longer than cell diameter in length (FIG. 6G), and the longest neurite (FIG. 6H) [23]. Only MNs with neurites present were included in analysis for longest neurite length (n=114 cells for BoTX-A, n=107 cells for SHAM). The proportion of MNs with initial process formation in the BoTX-A group was 0.86±0.02, which was significantly greater (p<0.001) than the SHAM group (0.60±0.04; FIG. 6F). The mean number of neurites per MNs was significantly higher (p<0.001) in the BoTX-A group, 1.61±0.11, as compared to the SHAM group, 0.86±0.08 (FIG. 6G). Finally, the mean length of the longest neurite after BoTX-A was 19.1±0.8 μm and SHAM was 11.1±0.7 μm, which was significantly different (p<0.001; FIG. 6H). These findings identified a direct impact on MN-neurite outgrowth that mirrors the enhanced outgrowth observed for the in vivo murine model of axon regeneration.

While the invention has been described in terms of various embodiments, it is understood that variations and modifications will occur to those skilled in the art. Therefore, it is intended that the appended claims cover all such equivalent variations that come within the scope of the invention as claimed. In addition, the section headings used herein are for organizational purposes only and are not to be construed as limiting the subject matter described.

Each embodiment herein described may be combined with any other embodiment or embodiments unless clearly indicated to the contrary. In particular, any feature or embodiment indicated as being preferred or advantageous may be combined with any other feature or features or embodiment or embodiments indicated as being preferred or advantageous, unless clearly indicated to the contrary.

All references cited in this application are expressly incorporated by reference herein.

### REFERENCES

[1] Kurtzke J F. The current neurologic burden of illness and injury in the United States. Neurology. 1982 November; 32(11):1207-14.
[2] Novak C B, Anastakis D J, Beaton D E, Katz J. Patient-reported outcome after peripheral nerve injury. J Hand Surg Am. 2009 February; 34(2):281-7.
[3] Fu S Y, Gordon T. Contributing factors to poor functional recovery after delayed nerve repair: prolonged denervation. J Neurosci. 1995 May; 15(5 Pt 2):3886-95.
[4] Gordon T, Tyreman N, Raji M A. The basis for diminished functional recovery after delayed peripheral nerve repair. J Neurosci. 2011 Apr. 6; 31(14):5325-34.
[5] Wong J N, Olson J L, Morhart M J, Chan K M. Electrical stimulation enhances sensory recovery: a randomized controlled trial. Ann Neurol. 2015 June; 77(6):996-1006.
[6] Gordon T, Amirjani N, Edwards D C, Chan K M. Brief post-surgical electrical stimulation accelerates axon regeneration and muscle reinnervation without affecting the functional measures in carpal tunnel syndrome patients. Exp Neurol. 2010 May; 223(1):192-202.
[7] Hoffman P N. A conditioning lesion induces changes in gene expression and axonal transport that enhance regeneration by increasing the intrinsic growth state of axons. Exp Neurol. 2010 May; 223(1):11-8.
[8] McQuarrie I G. The effect of a conditioning lesion on the regeneration of motor axons. Brain Res. 1978 Sep. 8; 152(3):597-602.
[9] McQuarrie I G, Grafstein B, Gershon M D. Axonal regeneration in the rat sciatic nerve: effect of a conditioning lesion and of dbcAMP. Brain Res. 1977 Sep. 2; 132(3):443-53.
[10] Garg R, Merrell G A, Hillstrom H J, Wolfe S W. Comparison of nerve transfers and nerve grafting for traumatic upper plexus palsy: a systematic review and analysis. J Bone Joint Surg Am. 2011 May 4; 93(9):819-29.
[11] Tung T H, Mackinnon S E. Nerve transfers: indications, techniques, and outcomes. J Hand Surg Am. 2010 February; 35(2):332-41.
[12] Carli L, Montecucco C, Rossetto O. Assay of diffusion of different botulinum neurotoxin type a formulations injected in the mouse leg. Muscle Nerve. 2009 September; 40(3):374-80.
[13] Hassan S M, Jennekens F G, Wieneke G, Veldman H. Calcitonin gene-related peptide-like immunoreactivity, in botulinum toxin-paralysed rat muscles. Neuromuscul Disord. 1994 Sep.-Nov. 4(5-6):489-96.
[14] Hassan S M, Jennekens F G, Veldman H, Oestreicher B A. GAP-43 and p75NGFR immunoreactivity in presynaptic cells following neuromuscular blockade by botulinum toxin in rat. J Neurocytol. 1994 June; 23(6):354-63.
[15] Bonner P H, Friedli A F, Baker R S. Botulinum A toxin stimulates neurite branching in nerve-muscle cocultures. Brain Res Dev Brain Res. 1994 May 13; 79(1):39-46.
[16] Pinter M J, Vanden Noven S, Muccio D, Wallace N. Axotomy-like changes in cat motoneuron electrical properties elicited by botulinum toxin depend on the complete elimination of neuromuscular transmission. J Neurosci. 1991 March; 11(3):657-66.
[17] Brooks V B. An intracellular study of the action of repetitive nerve volleys and of botulinum toxin on miniature end-plate potentials. J Physiol. 1956 Nov. 28; 134(2):264-77.
[18] Franz C K, Rutishauser U, Rafuse V F. Polysialylated neural cell adhesion molecule is necessary for selective targeting of regenerating motor neurons. J Neurosci. 2005 Feb. 23; 25(8):2081-91.
[19] Franz C K, Rutishauser U, Rafuse V F. Intrinsic neuronal properties control selective targeting of regenerating motoneurons. Brain. 2008 June; 131(Pt 6):1492-505.
[20] Abercrombie M. Estimation of nuclear population from microtome sections. Anat Rec. 1946 February; 94:239-47.
[21] Aoki K R. A comparison of the safety margins of botulinum neurotoxin serotypes A, B, and F in mice. Toxicon. 2001 December; 39(12):1815-20.
[22] Santos D P, Kiskinis E. Generation of Spinal Motor Neurons from Human Pluripotent Stem Cells. Methods Mol Biol. 2017; 1538:53-66.
[23] Singh B, Xu Q G, Franz C K, Zhang R, Dalton C, Gordon T, et al. Accelerated axon outgrowth, guidance, and target reinnervation across nerve transection gaps following a brief electrical stimulation paradigm. J Neurosurg. 2012 March; 116(3):498-512.
[24] Mackinnon S E. Donor Distal, Recipient Proximal and Other Personal Perspectives on Nerve Transfers. Hand Clin. 2016 May; 32(2):141-51.
[25] Duchen L W. Changes in motor innervation and cholinesterase localization induced by botulinum toxin in skeletal muscle of the mouse: differences between fast and slow muscles. J Neurol Neurosurg Psychiatry. 1970 February; 33(1):40-54.
[26] Morbiato L, Carli L, Johnson E A, Montecucco C, Molgo J, Rossetto O. Neuromuscular paralysis and recovery in mice injected with botulinum neurotoxins A and C. Eur J Neurosci. 2007 May; 25(9):2697-704.
[27] Duchen L W, Strich S J. The effects of botulinum toxin on the pattern of innervation of skeletal muscle in the mouse. Q J Exp Physiol Cogn Med Sci. 1968 January; 53(1):84-9.
[28] Pastor A M, Moreno-Lopez B, De La Cruz R R, Delgado-Garcia J M. Effects of botulinum neurotoxin type A on abducens motoneurons in the cat: ultrastructural and synaptic alterations. Neuroscience. 1997 November; 81(2):457-78.
[29] Fu S Y, Gordon T. The cellular and molecular basis of peripheral nerve regeneration. Mol Neurobiol. 1997 Feb.-Apr. 14(1-2):67-116.
[30] Chipman P H, Franz C K, Nelson A, Schachner M, Rafuse V F. Neural cell adhesion molecule is required for stability of reinnervated neuromuscular junctions. Eur J Neurosci. 2010 Jan. 31(2):238-49.
[31] Magill C K, Tong A, Kawamura D, Hayashi A, Hunter D A, Parsadanian A, et al. Reinnervation of the tibialis anterior following sciatic nerve crush injury: a confocal microscopic study in transgenic mice. Exp Neurol. 2007 September; 207(1):64-74.
[32] Brown M C, Hopkins W G. Role of degenerating axon pathways in regeneration of mouse soleus motor axons. J Physiol. 1981 September; 318:365-73.
[33] Duchen L W. The effects in the mouse of nerve crush and regneration on the innervation of skeletal muscles paralysed by Clostridium botulinum toxin. J Pathol. 1970 September; 102(1):9-14.
[34] Akdeniz Z D, Bayramicli M, Ates F, Ozkan N, Yucesoy C A, Ercan F. The role of botulinum toxin type a-induced motor endplates after peripheral nerve repair. Muscle Nerve. 2015 September; 52(3):412-8.
[35] Arntz C, Kanje M, Lundborg G. Regeneration of the rat sciatic nerve after different conditioning lesions: effects of the conditioning interval. Microsurgery. 1989; 10(2):118-21.
[36] Forman D S, McQuarrie I G, Labore F W, Wood D K, Stone L S, Braddock C H, et al. Time course of the conditioning lesion effect on axonal regeneration. Brain Res. 1980 Jan. 20; 182(1):180-5.
[37] Coffield J A, Yan X. Neuritogenic actions of botulinum neurotoxin A on cultured motor neurons. J Pharmacol Exp Ther. 2009 July; 330(1):352-8.
[38] Osen-Sand A, Staple J K, Naldi E, Schiavo G, Rossetto O, Petitpierre S, et al. Common and distinct fusion proteins in axonal growth and transmitter release. J Comp Neurol. 1996 Apr. 1; 367(2):222-34.
[39] Morihara T, Mizoguchi A, Takahashi M, Kozaki S, Tsujihara T, Kawano S, et al. Distribution of synaptosomal-associated protein 25 in nerve growth cones and reduction of neurite outgrowth by botulinum neurotoxin A without altering growth cone morphology in dorsal root ganglion neurons and PC-12 cells. Neuroscience. 1999; 91(2):695-706.
[40] Hug A, Weidner N. From bench to beside to cure spinal cord injury: lost in translation? Int Rev Neurobiol. 2012; 106:173-96.
[41] Kiskinis E, Eggan K. Progress toward the clinical application of patient-specific pluripotent stem cells. J Clin Invest. 2010 January; 120(1):51-9.
[42] Tos P, Ronchi G, Papalia I, Sallen V, Legagneux J, Geuna S, et al. Chapter 4: Methods and protocols in peripheral nerve regeneration experimental research: part I-experimental models. Int Rev Neurobiol. 2009; 87:47-79.
[43] McQuarrie I G, Jacob J M. Conditioning nerve crush accelerates cytoskeletal protein transport in sprouts that form after a subsequent crush. J Comp Neurol. 1991 Mar. 1; 305(1):139-47.
[44] Neumann S, Bradke F, Tessier-Lavigne M, Basbaum A I. Regeneration of sensory axons within the injured spinal cord induced by intraganglionic cAMP elevation. Neuron. 2002 Jun. 13; 34(6):885-93.
[45] Qiu J, Cai D, Dai H, McAtee M, Hoffman P N, Bregman B S, et al. Spinal axon regeneration induced by elevation of cyclic AMP. Neuron. 2002 Jun. 13; 34(6):895-903.
[46] Gracies J M, Brashear A, Jech R, McAllister P, Banach M, Valkovic P, et al. Safety and efficacy of abobotulinumtoxinA for hemiparesis in adults with upper limb spasticity after stroke or traumatic brain injury: a double-blind randomised controlled trial. Lancet Neurol. 2015 Oct. 14(10):992-1001.
[47] Kaji R, Osako Y, Suyama K, Maeda T, Uechi Y, Iwasaki M, et al. Botulinum toxin type A in post-stroke lower limb spasticity: a multicenter, double-blind, placebo-controlled trial. J Neurol. 2010 August; 257(8):1330-7.
[48] Bakheit A M, Thilmann A F, Ward A B, Poewe W, Wissel J, Muller J, et al. A randomized, double-blind, placebo-controlled, dose-ranging study to compare the efficacy and safety of three doses of botulinum toxin type A (Dysport) with placebo in upper limb spasticity after stroke. Stroke. 2000 October; 31(10):2402-6.
[49] Nalysnyk L, Papapetropoulos S, Rotella P, Simeone J C, Alter K E, Esquenazi A. OnabotulinumtoxinA muscle injection patterns in adult spasticity: a systematic literature review. BMC Neurol. 2013 Sep. 8; 13:118.
[50] Fox I K, Davidge K M, Novak C B, et al. Nerve transfers to restore upper extremity function in cervical spinal cord injury: update and preliminary outcomes. Plast Reconstr Surg. 2015 October; 136(4):780-92.
[51] Zheng M. X, M. D., X. Y Hua, J. T Feng, et al. Trial of contralateral seventh cervical nerve transfer for spastic arm paralysis. N Engl J Med 2018; 378:22-34.

1. A method of inducing growth of a peripheral nervous system neuron, the method comprising administering to the neuron an effective amount of a botulinum toxin.

2. The method of claim 1, wherein the peripheral nervous system neuron is a motor neuron.

3. The method of claim 1, wherein the neuron has been damaged.

4. The method of claim 1, wherein the neuron has been axotomized.

5. The method of claim 1, wherein the neuron growth comprises neurite outgrowth.

6. The method of claim 1, wherein the neuron growth comprises neuronal regeneration.

7. The method of claim 1, wherein the neuron is in a mammal.

8. The method of claim 1, wherein the neuron is in a human.

9. The method of claim 1, wherein the effective amount of the botulinum toxin is 0.1 units (4.4-5.1 units/kg), 0.25 units (11.0-12.75 units/kg) or 0.5 units (22.0-25.5 units/kg).

10. The method of claim 9, wherein the effective amount of the botulinum toxin is 0.25 units (11.0-12.75 units/kg).

11. The method of claim 1, wherein the botulinum toxin is administered to the neuron by injection.

12. The method of claim 1, wherein the botulinum toxin is administered topically to the neuron.

13. The method of claim 1, wherein the botulinum toxin is administered to the cell body of the neuron.

14. The method of claim 1, wherein the botulinum toxin is administered the axon of the neuron.

15. The method of claim 1, wherein the botulinum toxin is type A neurotoxin.

16. A method of inducing neuronal growth in a patient in need thereof, the method comprising, administering to the patient an effective amount of botulinum toxin.

17. The method of claim 16, wherein the patient is undergoing a nerve transfer surgery.

18. The method of claim 17, wherein the botulinum toxin is administered to the patient one week prior to surgery.

19. The method of claim 16, wherein the neuron growth comprises neurite outgrowth.

20. The method of claim 16, wherein the neuron growth comprises neuronal regeneration.
